(19) United States
(12) Patent Application Publication
(10) Pub. No.: US 2005/0042363 A1
(43) Pub. Date: Feb. 24, 2005

(54) Method for fabrication of biochips with a macroporous polymer substrate

(76) Inventors: Alexander V. Kukhtin, Lockport, IL (US); Boris K. Chernov, Willowbrook, IL (US); Julia B. Golova, Willowbrook, IL (US); Gennadiy M. Yershov, Willowbrook, IL (US); Margaret Anne Gemmell, Western Springs, IL (US)

Correspondence Address: Alice O. Martin, Barnes & Thornburg, P.O. Box 2786, Chicago, IL, 60690-2809, US

(21) Appl. No.: 10/642,768

(22) Filed: Aug. 18, 2003

(51) Int. Cl. B05D 3/00; A61L 2/00

(52) U.S. Cl. 427/8; 427/2.1

(57) ABSTRACT

Macroporous polymer substrates have greater immobilization capacity for large biomolecules and better access for analytes than substrates used previously for microarrays. Microarrays are fabricated with macroporous polymer substrates, and nucleic acids, proteins and peptides are immobilized in the substrate. Microarrays with macroporous polymer substrates are useful in immunoassays, drug discovery studies and other biotechnological applications that involve large scale macromolecular interactions. Nucleic acid hybridizations, protein binding, and antigen-antibody interactions are analyzed using microarrays with macroporous polymer substrates.

The United States Government has rights in this invention pursuant to Contract No. W-31-109-ENG-38 between the U.S. Department of Energy (DOE) and the University of Chicago representing Argonne National Laboratory

Macroporous polymer substrates are fabricated for microarrays.

### BACKGROUND

Several different types of assays use microarrays containing different types of immobilized biological molecules. For example, hybridization of nucleic acids on biochips that are imprinted with oligonucleotides or cDNAs are used for detection of large scale gene expression profiles, identification of mutations, detection of single nucleotide polymorphisms and detection of different microbial species. Biochips with immobilized proteins or peptides are useful for immunoassays, drug discoveries, and investigations of macromolecular interactions. Thus, different types of biochips are used in areas of medicine, biotechnology, and biology. Parallel detection of hundreds and thousands of interactions between a biological sample of interest with immobilized molecules on a biochip provides powerful tools to investigate complex biological interactions that involve large molecules.

The most commonly used substrate for attachment of biological compounds to biochips is glass. The surface of glass slides is usually modified to provide either chemical or physical attachment of biomolecules. Some biochips use glass surfaces modified with a polymer layer or a gel array template. Such a polymer layer is either microporous polymer film such as Nylon (Atlas arrays from Clontech, Palo Alto, Calif.) or expandable hydrogels such as polyacrylamide or agarose compositions (Hydrogel substrates from Perkin-Elmer, Wellesley, Mass.; Nanochip from Nanogen, San Diego, Calif.). In comparison with the biochips that use flat surfaces as substrates, the hydrogels provide increased immobilization capacity and better microenvironment for immobilized biomolecules, especially for proteins, to achieve better sensitivity and detection limits.

However, one of the problems encountered with hydrogels is the poor accessibility of analyte (target) to immobilized biomolecules (probes) when compared with a flat surface. The accessibility of analytes becomes critical for assays with large biomolecules, e.g., immunoassays involving large proteins or peptides, where each assay step takes about 1 hour. Thus, microarrays that can accommodate large molecules and efficiently detect interactions involving large molecules are needed in diagnostics, pharmaceutics, and other biotechnological applications.

### SUMMARY OF THE INVENTION

Methods and compositions are presented for fabrication of microarrays with macroporous polymer substrates that have high immobilization capacity for large biomolecules and better accessibility of analytes to the immobilized biomolecules. Microarrays with macroporous polymer substrates can be stored in room temperature for several months prior to immobilization of biomolecules on them.

A macroporous polymer substrate includes:
a methacrylate;
a dimethacrylate (tri-, tetramethacrylate;
an immobilization chemical; and
a polymer solvent.

A method of making a microarray with a macroporous polymer substrate includes the steps of:
obtaining a macroporous polymer substrate; and
coating a surface with the macroporous polymer substrate.

The macroporous polymer substrate is synthesized by:
obtaining methacrylates and mixing the methacrylates in the presence of a porogenic solvent to form a macroporous polymer substrate.

Methacrylates may be monofuncional or polyfunctional. Suitable methacrylates include GMA, HEMA, EDMA, and DHDM. A suitable methacrylate includes glycidyl methacrylate, and 2-hydroxyethyl methacrylate. Monofunctional methacrylate monomers are alkyl-, epoxyalkyl-, hydroxyalkyl-, and polyoxyalkyl ethers of methacrylic acid. Polyfunctional methacrylate monomers (cross-linking monomers) are dimethacrylates of ethylene glycol or di-, tri, or tetramethacrylates of polyols. Suitable dimethacrylates include ethylene dimethacrylate, and 2,3-dihydroxybutane-1,4-diyl dimethacrylate.

Concentration of monofunctional acrylates in the polymerization mix is 4-30%, concentration of cross-linking di-, tri-, or tetramethacrylates is 2-20%, concentration of immobilization chemical is 0-5%, concentration of the solvent is 50-94% with various ratios between solvent components.

A suitable porogenic solvent is an aromatic alcohol, e.g. cyclohexanol and dodecanol. Porogenic solvents may include a mix of cyclohexanol, different aliphatic alcohols and may also contain aromatic alkyl derivatives (e.g., toluene, xylene).

Another porogenic solvent is an aliphatic alcohol.

Polymerization may be initiated by light in the presence of such photoinitiators as benzoin, benzoin methyl ether, benzoin ethyl ether, and 2,2-dimethoxy-2-phenyl-acetophenone. Polymerization may also be initiated by heating polymerization mix in the presence of chemical initiator such as azoisobuyronitril.

Suitable biomolecules include DNA, RNA, peptides, proteins, lipids, lipopolysaccharides, antibodies, and peptide mimetics.

Biomolecules are immobilized. An immobilization chemical for immobilization of biomolecules may be added to the microarray. A suitable immobilization chemical includes N-(methacryloyl)aminocaproic acid N-hydroxy succinimide ether, 4-isothiocyanate-N-(methacryloyl)benzylamine, and N-(5,6-di-O-isopropylidene)hexyl acrylamide.

A suitable surface includes glass, metal, silicone, and different plastics provided with vinyl groups. The macroporous polymer can also be used as an array substrate without any external support.

An immobilization chemical is derivatized to include functional groups such as aldehydes, succinimides and isothiocyanates.

A suitable immobilization chemical includes N-(methacryloyl)aminocaproic acid N-hydroxysuccinimide ether, 4-isothiocyanate-N-(methacryloyl)benzylamine, and N-(5,6-di-O-isopropylidene)hexyl acrylamide.

Embodiments of the macroporous polymer substrate include:
(a) 4-30% GMA, 2-20% of EDMA, 0-5% MAAHSE, 48-60% cyclohexanol, and 0-12% dodecanol.
(b) 4-30% GMA, 2-20% of DHDM, 0-5% ITCMBA, 48-60% cyclohexanol, and 0-12% dodecanol.
(c) 4-30% GMA, 2-20% of EDMA, 0-5% ITCMBA, 48-60% cyclohexanol, and 0-12% dodecanol.
(d) 4-30% GMA, 2-20% of DHDM, 0-5% MAAHSE, 48-60% cyclohexanol, and 0-12% dodecanol.
(e) 4-30% HEMA, 2-20% of EDMA, 0-5% MAAHSE, 48-60% cyclohexanol, and 0-12% dodecanol.
(f) 4-30% HEMA, 2-20% of DHDM, 0-5% ITCMBA, 48-60% cyclohexanol, and 0-12% dodecanol.
(g) 4-30% HEMA, 2-20% of EDMA, 0-5% ITCMBA, 48-60% cyclohexanol, and 0-12% dodecanol.
(h) 4-30% HEMA, 16% of DHDM, 0-5% MAAHSE, 48-60% cyclohexanol, and 0-12% dodecanol.
   Definitions

Analyte
a target molecule.

Array, Microarray
molecules connected to a matrix or support in a specific arrangement relative to each other, also known as DNA microarray, DNA array or peptide array

Biochip
Biochip is a set of (array of) biological molecules (called probes) attached in an appropriate order to a substrate/support or matrix. Also known as a chip, DNA chip or peptide chip; includes array of biological molecules such as DNA fragments, peptides, proteins, lipids, and tissues connected to a substrate.

Biological sample
a biological material obtained from tissues and organs, e.g. saliva, tears, bodily fluids or bodily secretions, liver, skin, blood; obtained from other organisms such as bacteria.

Bioprobe, probe
molecule which can be used to identify or characterize another molecule e.g. by hybridizing or binding. Usually a molecule immobilized on a biochip.

Coating
a covering.

Compound I
MAAHSE (N-(methacryloyl)aminocaproic acid N-hydroxy succinimide ether.

Compound II
(ITCMBA) 4-isothiocyanate-N-(methacryloyl)benzylamine.

Compound III
N-(5,6-di-O-isopropylidene)hexyl acrylamide.

Derivatization
conversion of chemical groups present in the polymer to functional groups. Also known as functionalization.

DHDM
2,3-dihydroxybutane-1,4-diyl dimethacrylate.

EDMA
ethylene dimethacrylate.

Functional groups
chemically active moieties present in the substrate, probe, or analyte capable of reacting and forming a covalent linkage.

Functionalization
conversion of chemical groups present in the polymer to functional groups. Also known as derivatization.

GMA
glycidyl methacrylate.

Gel array template
An array of gel (polymer) elements (pads). Also referred herein as microarray.

HEMA
2-hydroxyethyl methacrylate

Hybridization
the formation of duplex molecules from complementary single strands (e.g., DNA-DNA, DNA-RNA, RNA-RNA). A single stranded nucleic acid molecule is generally labeled, e.g. with a detectable dye (radioactive or fluorescent) and used as a probe that may anneal to molecules with similar sequences that are single stranded. Conditions are varied to detect degrees of similarity, i.e. the more stringent the conditions, the greater the similarity needed for hybridization to occur.

Hydrophilicity
affinity for water.

Immobilization
fixation of biomolecules onto a matrix or substrate.

Immobilization chemical
chemical used to attach biomolecules onto to a matrix chemical through chemical modifications.

MAASHE
N-(methacryloyl)aminocaproic acid N-hydroxysuccinimide ether.

Macroporous
interstitial voids sufficient to accommodate large molecules such as proteins, DNA, RNA, peptides and antibodies.

Matrix
a support such as glass slide, modified glass slide (e.g. silanized) silicon, gold slide, gel pad, nylon membrane or other similar structures on which an array or microarray of molecules is formed. A matrix or support may contain functional groups to attach biomolecules.

Monofunctional methacrylate
methacrylates having such as alkyl-, epoxyalkyl-, hydroxyalkyl-, and polyoxyalkyl ethers of methacrylic acid.

Nuclei
insoluble, gel-like species.

Peptide mimetic
peptide that biologically mimics active determinants on hormones, cytokines, enzyme substrates, viruses or other biomolecules, and may antagonize, stimulate, or otherwise modulates the physiological activity of the natural ligands.

Photopolymerization
Polymerization initiated by light in the presence of photoinitiators such as benzoin, benzoin methyl ether, benzoin ethyl ether, and 2,2-dimethoxy-2-phenyl-acetophenone.

Polyfunctional methacrylate
include methacrylate monomers (cross-linking monomers) such as dimethacrylates of ethylene glycol or di-, tri, or tetramethacrylates of polyols.

Porogenic
thermodynamically a poor solvent—a solvent that contributes to pore formation during polymerization. Solubility is good for initial monomers but poor for forming polymer—this phenomenon results in the precipitation of forming polymer particles. Porogenic solvent could be a mix of cyclohexanol, different aliphatic alcohols and may contain aromatic alkyl derivatives (e.g., toluene, xylene).

Protein/peptide array, protein/peptide chip
a solid or semi-solid support onto which several proteins or peptides are spotted in an ordered fashion to allow visualization and analysis of protein-protein (e.g, antigen-antibody), protein-peptide, peptide-peptide, and protein-ligand interactions. The support can be in the form of a glass slide, glass slide coated with a suitable polymer, or hydrogels.

Substrate
a solid, semi-solid, or a liquid support onto which biomolecules can be attached for analysis.

Transparency
a physico-chemical characteristic of a polymer.

### BRIEF DESCRIPTION OF THE DRAWINGS

FIG. 1 illustrates the chemical structures of monofunctional monomers glycidyl methacrylate (GMA) and 2-hydroxyethyl methacrylate (HEMA) for macroporous polymer substrate fabrication.

FIG. 2 illustrates the chemical structures of bifunctional monomers ethylene dimethacrylate (EDMA) and 2,3-dihydroxybutane-1,4-diyl dimethacrylate (DHDM).

FIG. 3 illustrates the chemical structures of immobilization chemicals N-(methacryloyl)aminocaproic acid N-hydroxy succinimide ether (I) and 4-isothiocyanate-N-(methacryloyl)benzylamine (II).

FIG. 4 shows the hybridization signals obtained with different substrates. 1—GMA-EDMA with I; 2—GMA-EDMA without I or II or III; 3—Standard acrylamide with III. AU-arbitrary units measured relative to the control.

FIG. 5 shows the efficiency of binding for Streptavidin-Texas Conjugate with biotinylated BSA on GMA-DHDM and commercially available 3D biochip platforms. Biotinylated BSA (1 mg/ml) (2 replicates) were immobilized onto different biochip substrates. Biochips were reacted with 10 ng/ml (A) and 1 μg/ml (B) Streptavidin-Texas Conjugate for 1.5 h. Fluorescent intensities were recorded on Biochip Images scanner (Packard Bioscience, Boston, Mass.). The average signal intensity for empty spots was calculated and then subtracted from the average signal intensity for spots with biotinylated BSA. AU-arbitrary units measured relative to the control.

FIG. 6 shows the logarithmic dependence of fluorescent signal on the IL-1β cytokine concentration during an immunoassay. AU-arbitrary units measured relative to the control.

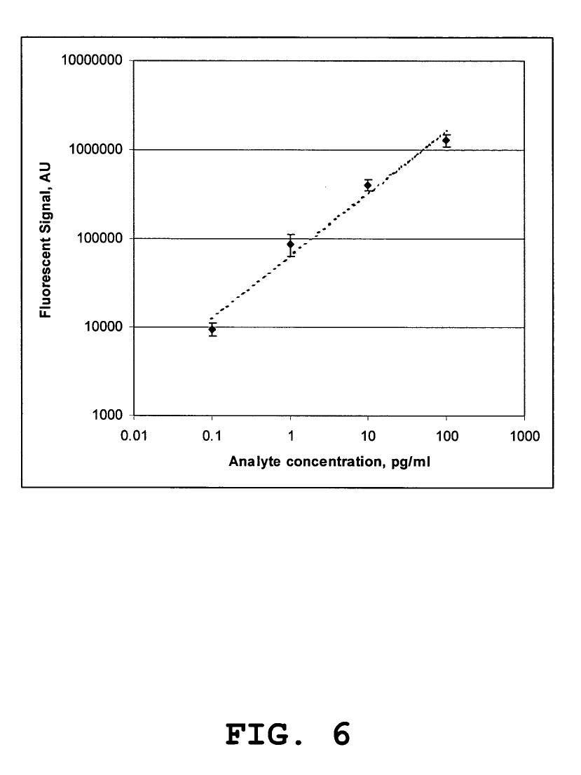

FIG. 7 shows the binding of goat anti-mouse-Texas Red conjugate with different protein probes on GMA-EDMA, GMA-DHDM, polyacrylamide and SuperAldehyde substrates. Two mouse monoclonal antibodies, M421AE and M620E, and Protein A (all at concentrations of 100 mg/ml) (10 replicates for each protein probe) were immobilized onto different biochip substrates. Biochips were exposed to 1 μg/ml goat-antimouse-Texas Conjugate for 1.5 h. Fluorescent intensities were recorded on a fluorescent microscope. Average fluorescent values for empty spots were calculated and then subtracted from the average values for spots with the protein probes for each biochip.

### DETAILED DESCRIPTION OF THE INVENTION

A microarray (biochip) substrate with high capacity and accessibility for immobilized biomolecules is produced by using polymerization of mono- and bi-, tri-, poly-functional acrylate, methacrylate, and other vinyl derivatives in the presence of a porogenic solvent or porogenic agents. Basic mechanisms of pore formation are described in Svec and Frechet (1995), Naghash et al. (1997), and Horak and Labsky (1997).

Monofunctional monomers such as GMA, HEMA and bifunctional monomers such as EDMA, DHDM are used to fabricate macroporous polymer substrates suitable for microarrays. As monomers form polymers during radical polymerization, polymers become insoluble in the reaction medium in the presence of a thermodynamically poor solvent (porogen) and precipitate to form insoluble gel-like species (nuclei). Further polymerization proceeds both in solution and within swollen nuclei where it is kinetically preferred because local concentration of monomers is higher in the nuclei than in solution. Growing nuclei associate in clusters that form a scaffolding-like interconnected matrix on later stages of the polymerization. The interconnected matrix gets reinforced by both inter-globular cross-linking and the capture of chains that still polymerize in the solution phase during continuing polymerization leading to the final porous polymer body. The fraction of voids (macropores) within the final porous polymer is close to the volume fraction of the porogenic solvent in the initial polymerization mix because the porogen remains trapped in the voids of the cross-linked polymer. Change in the nature and concentration of initial monomers, porogen, reaction temperature, and initiator of a radical polymerization allows production of polymer structures with a wide variety of average pore size (1-1000 nm) and physico-chemical properties such as transparency, hydrophilicity, and density. This allows control of polymer size to enable custom fabrication of substrates for microarrays designed to analyze complex biological molecules, e.g. proteins having a molecular weight of 150 kDa are analyzed with the macroporous polymer substrate.

However, in order to use the above-described macroporous polymer substrates for biochip fabrication, they should be derivatized with chemically-reactive groups specific for chemical groups that are present in biological molecules to be immobilized on a biochip (e.g., amino groups, carboxylic groups). This derivatization is achieved by incorporation of vinyl monomers that react after polymerization with some of the chemical groups from biomolecules to form covalent bonds. The derivatization is also achieved after polymerization, by incorporation of vinyl monomers with chemical groups that are converted to some other chemical groups that with the chemical groups from the biomolecules to form covalent bonds.

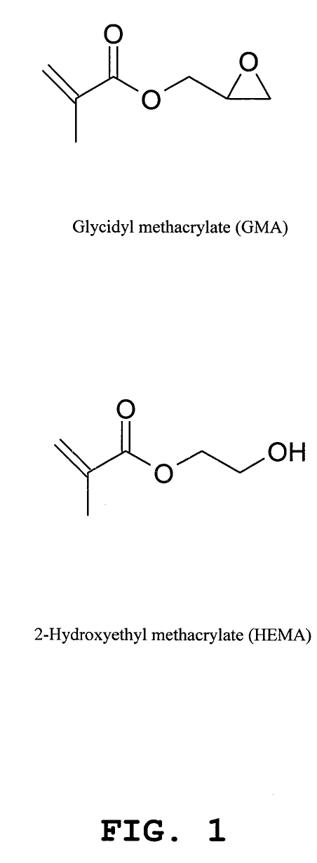

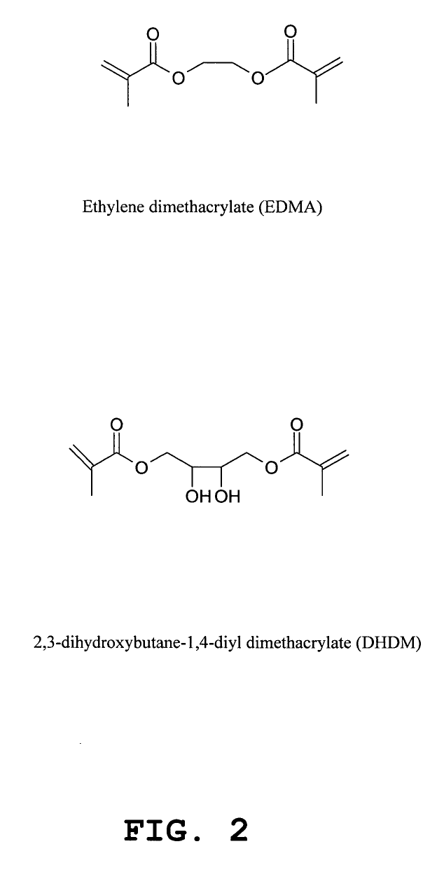

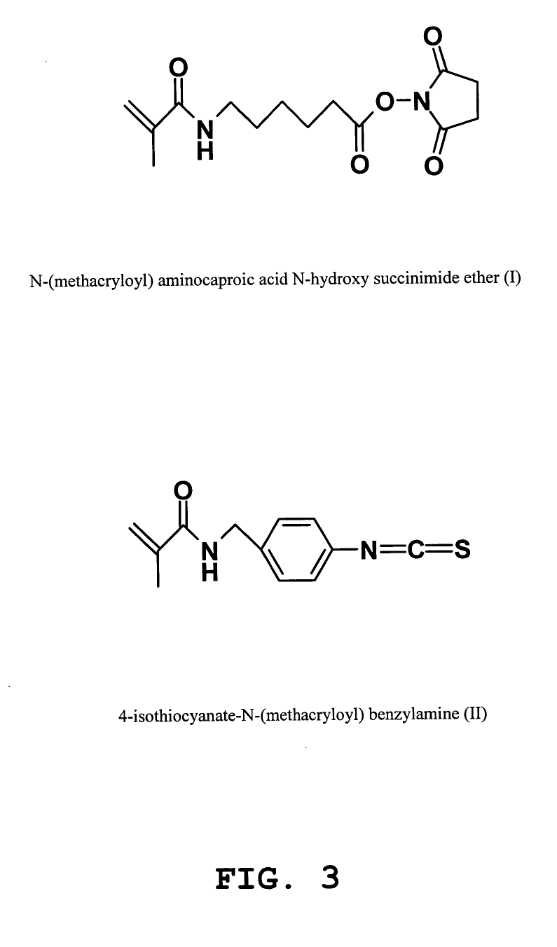

Macroporous biochip polymer substrates are produced by polymerization of glycidyl methacrylate (GMA) (FIG. 1) and 2-hydroxyethyl methacrylate (HEMA) (FIG. 1) taken in different ratios, and ethylene dimethacrylate (EDMA) (FIG. 2) and 2,3-dihydroxybutane, 1,4-diyl dimethacrylate (DHDM) (FIG. 2) taken in different ratios in the solvent containing cyclohexanol and dodecanol in different ratios. The polymerization mix may contain N-(methacryloyl)aminocaproic acid N-hydroxysuccinimide ether (I) (FIG. 3) or 4-isothiocyanate-N-(methacryloyl)benzylamine (II) (FIG. 3) or N-(5,6-di-O-isopropylidene)hexyl acrylamide (III) for substrate functionalization (U.S. Pat. No. 6,458,584).

Immobilization of proteins or peptides, or oligonucleotides modified with amino groups, is achieved by chemical coupling of I or II to the amino groups. Immobilization is also achieved by converting III to provide aldehyde groups.

Immobilization of proteins or peptides, or oligonucleotides is also performed on GMA biochip substrates without I or II or III, either by utilizing epoxide groups embedded into the polymer structure, or after converting the embedded epoxide groups to aldehyde groups. Epoxide groups present in the GMA biochip substrate are removed from the biochip substrate either prior to immobilization of the biomolecules on the biochip substrate or after the immobilization. On GMA biochip substrates with I and II, epoxide groups are removed after the immobilization by reacting the substrate with either amino-and/or sulfohydryl-containing compounds (e.g. Tris-HCl, cystein chloride). On GMA biochip substrates with III or without I, II or III epoxide groups are also removed prior to the immobilization of biomolecules by hydrolysis catalyzed by a Lewis acid (e.g., boron trifluride etherate).

Biochips fabricated with the macroporous polymer substrates are useful for biological assays with nucleic acids, peptides or proteins. The biochips with the substrates of the present invention are also used for complex immunoassays involving antibodies and antigens. The biochips produced with the macroporous polymer substrates possess higher capacity for large biomolecules and better access for analyte assays.

### EXAMPLES

### Example 1

### Synthesis of (I)

### a) Synthesis of N-Methacryloyl-6-aminocaproic acid

6-Aminocaproic acid (50 mmol, 6.56 g) was dissolved in 75 ml of glacial acetic acid, and methacrylic anhydride (55 mmol, 8.2 ml) was dropped into this solution. The reaction mixture was stirred under the room temperature during 48 hours and then evaporated in vacuo. The product obtained was purified by chromatography on silica gel column (4×30 cm) using chloroform-acetone (10:2) mixture as a mobile phase. The collected fractions were evaporated to dryness to obtain compound N-Methacryloyl-6-aminocaproic acid at a yield of 88%.

C₁₀H₁₇NO₃ MS: calc. 199.4, found 200.5.

### b) Synthesis of (I) from N-methacryloyl-6-aminocaproic acid

N-Methacryloyl-6-aminocaproic acid (24.6 mmol, 4.9 g) was dissolved in 80 ml of acetone. After that N,N-dicyclohexylcarbodiimide (27.0 mmol, 5.57 g) and N-hydroxysuccinimide (27.0 mmol, 3.1 g) were added to the solution. The reaction mixture was stirred at room temperature overnight. The formed precipitate was filtered and washed with acetone, the filtrate was concentrated in vacuo. The resulting oil was taken up in ethyl acetate (100 ml) and this solution was sequentially washed with saturated aqueous solution of NaHCO₃ (2×50 ml), water (2×50 ml), dried over Na₂SO₄, filtered and concentrated to dryness. The residue was purified by silica gel column chromatography, which was performed with chloroform with applying gradient of acetone 1-5% to give (I) with yield 78%.

C₁₉H₁₈N₂O₄ MS: calc. 312.4, found 313.8.

### Example 2

### Synthesis of (II)

### a) Synthesis of 4-Amino-N-methacryloylbenzylamine

The solution of methacrylic anhydride (33.0 mmol, 4.9 ml) in 10 ml of tetrahydrofuran was slowly dropped under continuous stirring to the solution of 4-aminobenzylamine (30.0 mmol, 3.66 g) and triethylamine (30.0 mmol, 4.16 mg) in 25 ml of 2-propanol. The reaction was allowed to proceed at room temperature for two hours. After evaporation in vacuo, the oil residue was dissolved in 25 ml of chloroform and washed sequentially with saturated aqueous solution of NaHCO₃ (2×15 ml), water (2×15 ml), dried over Na₂SO₄, filtered and evaporated. The product obtained was purified by chromatography on silica gel column (2.5×20 cm) using mixture of chloroform-methanol (15:1) as a mobile phase. Collected fractions were concentrated to dryness to obtain 4-Amino-N-methacryloylbenzylamine with yield 67%. C₁₁H₁₄N₂O MS: calc. 190.9, found 191.9 λ max 240.8 nm (EtOH).

### b) Synthesis of (II) from 4-Amino-N-methacryloylbenzylamine

The solution of 4-amino-N-methacryloilbenzylamine (19.8 mmol, 3.77 g) in 40 ml of chloroform was added drop-wise to the solution of thiocarbonyldiimidazole (19.8 mmol, 3.53 g) in 60 ml of chloroform, and the mixture was stirred for 1.5 hours under room temperature. After completion of the reaction, the mixture was concentrated and the desired product was purified by silica gel column chromatography using chloroform as a mobile phase. The product was obtained with the yield 93.6%. C₁₂H₁₂N₂OS MS: calc. 232.3, found 233.7 λ max 269.7, 281.3 nm (MeOH).

### Example 3

### Synthesis of (III)

III was synthesized according to the procedure described in U.S. Pat. No. 6,458,584.

### Example 4

### Fabrication of Gel Array Templates with GMA-EDMA Substrates with I (II, III)

A polymerization chamber included a glass slide treated with Bind-Silane (Amersham-Pharmacia-Biotech, Piscataway, N.J.) and a quartz plate mask with specifications: transparent, 100×100 μm square windows, spaced by 200 μm, arranged on a 1 μmm thick chromium non-transparent layer. The slide and the mask were separated by 10 μm-thick spacers.

A polymerization mix was prepared according to the following method. Compound I or II or III was dissolved in GMA (Aldrich, St. Louis, Mo.) to prepare 20% (w/v) solution. 6 vol parts of the resulting solution was mixed with 4 vol parts of EDMA (Aldrich, St. Louis, Mo.). 4 vol parts of the resulting solution was mixed with 6 vol parts of cyclohexanol-dodecanol solution (9:1) (v/v). 3 μl of 10% (w/v) 2,2-dimethoxy-2-phenyl-acetophenone in cyclohexanol was added to 100 μl of the above-prepared solution prior to photopolymerization, the resulting mix was vortexed for 10-15 seconds and degassed for 3 minutes. The polymerization chamber was then filled with the polymerization mix and illuminated under Oriel Light Source (Oriel Instruments, Stratford, Conn., USA) for 15 minutes. After photopolymerization, the slide with the formed polymer template (matrix) was separated from the mask. The matrix was briefly washed with ethanol and water. Polymer debris that surrounded gel pads of the matrix was removed by silica suspension in water. The matrix was then washed in 30 min in methanol, then washed in 30 min in methanol-water solution (1:1, v/v) and dried.

### Example 5

### Fabrication of Gel Array Templates with GMA-DHDM Substrates with I (or II or III)

The polymerization chamber and the washing of the matrix were as described in Example 4.

A polymerization mix was prepared according to the following method. I (or II or III) was dissolved in GMA to prepare 20% (w/v) solution. DHDM was dissolved in the resulting mix to prepare 40% (w/v) solution. 4 vol parts of the resulting solution was mixed with 6 vol parts of cyclohexanol-dodecanol solution (9:1) (v/v). 1.5-3 μl of 10% (w/v) 2,2-dimethoxy-2-phenyl-acetophenone in cyclohexanol was added to 100 μl of the above prepared solution prior to photopolymerization. The resulting mix was vortexed for 10-15 s and degassed for 3 min. The polymerization chamber was then filled with the polymerization mix and illuminated under Oriel Light Source (Oriel Instruments, Stratford, Conn., USA) for 15 min. The assembly is disassembled under ethanol, the polymer substrate is washed under water and then ethanol. The polymeric/oligomeric debris remaining after photopolymerization were removed from the slide without destroying gel pads by wet abrasive slurry, e.g., 10 μm silica particles. Then the slide containing the polymer substrate was washed for 30 min in methanol and then for 30 min in methanol/water (50% v/v) to remove soluble oligomers. Then the slide containing the polymer substrate was dried in air and could be kept at least several months in dry place at room temperature before further application.

### Example 6

### Fabrication of Gel Array Templates with HEMA-EDMA Substrates with I (II, III)

The polymerization chamber and the washing of the matrix were as described in Example 4.

A polymerization mix was prepared according to the following method. I (II, III) was dissolved in GMA to prepare 20% (w/v) solution. 6 vol parts of the resulting solution was mixed with 4 vol parts of EDMA. 4 vol parts of the resulting solution was mixed with 6 vol parts of cyclohexanol-dodecanol solution (9:1) (v/v). 2 μl of 1% (w/v) 2,2-dimethoxy-2-phenyl-acetophenone in cyclohexanol was added to 100 μl of the above prepared solution prior to photopolymerization. The resulting mix was vortexed for 10-15 seconds and degassed for 3 minutes. The polymerization chamber was then filled with the polymerization mix and illuminated under Oriel Light Source for 15 minutes.

### Example 7

### Fabrication of Gel Array Templates with HEMA-DHDM Substrates with I (II, III)

The polymerization chamber, polymerization procedure and washing of the matrix were as described in Example 4.

A polymerization mix was prepared according to the following method. I (or II or III) was dissolved in GMA (Aldrich) to prepare 20% (w/v) solution. DHDM was dissolved in the resulting mix to prepare 40% (w/v) solution. 4 vol parts of the resulting solution was mixed with 6 vol parts of cyclohexanol-dodecanol solution (9:1) (v/v). 2 μl of 1% (w/v) 2,2-dimethoxy-2-phenyl-acetophenone in cyclohexanol was added to 100 μl of the above prepared solution prior to photopolymerization, the resulting mix was vortexed 10-15 s and degassed for 3 min. The polymerization chamber was then filled with the polymerization mix and illuminated under Oriel Light Source for 5 min.

### Example 8

### Fabrication of Gel Array Templates with GMA(HEMA)-EDMA(DHDM) Substrates without I (or II or III)

A GMA (HEMA)-EDMA (DHDM) substrate without I (or II or III) was produced according to Examples 3-6, except that pure GMA (HEMA) was used for the preparation of the polymerization mix instead of 20% solution of I (or II or III) in GMA (HEMA).

### Example 9

### Fabrication of Gel Array Templates with GMA-DHDM Substrates without I (or II or III)

The polymerization chamber and the washing of the matrix were as described in Example 4.

A polymerization mix was prepared according to the following method. I (or II or III) was dissolved in GMA (Aldrich) to prepare 20% (w/v) solution. DHDM was dissolved in the resulting mix to prepare 40% (w/v) solution. 4 vol parts of the resulting solution was mixed with 6 vol parts of toluene (v/v). 9 μl of 10% (w/v) 2,2-dimethoxy-2-phenyl-acetophenone in cyclohexanol was added to 100 μl of the above prepared solution prior to photopolymerization. The resulting mix was vortexed for 10-15 seconds and degassed for 3 minutes. The polymerization chamber was then filled with the polymerization mix and illuminated under Oriel Light Source for 15 minutes.

### Example 10

### Providing Aldehyde Groups to Biochip Substrates Produced with GMA but without I (or II or III)

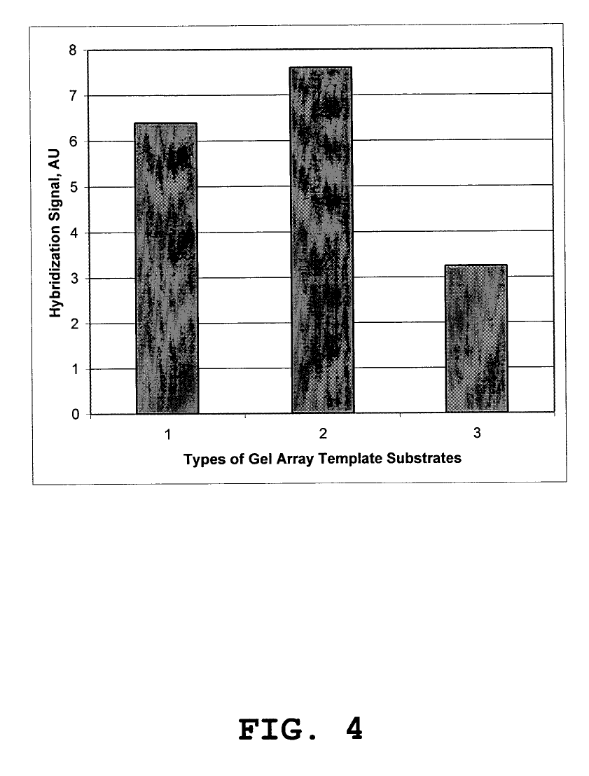

A biochip substrate produced with GMA but without I (or II or III), as described above, was incubated for 3 hours in 0.5 M boron trifluoride etherate in ethanol-water (9:1 v/v) to activate epoxide groups of GMA incorporated into the polymer. The substrate was then washed with deionized ethanol for 5 min and then with water for 5 min. Then the substrate was treated with 0.1 M sodium periodate for 30 min at room temperature to produce aldehyde groups (FIG. 4). After the treatment, the substrate was air dried.

### Example 11

### Providing Aldehyde Groups to Biochip Substrates Produced with III

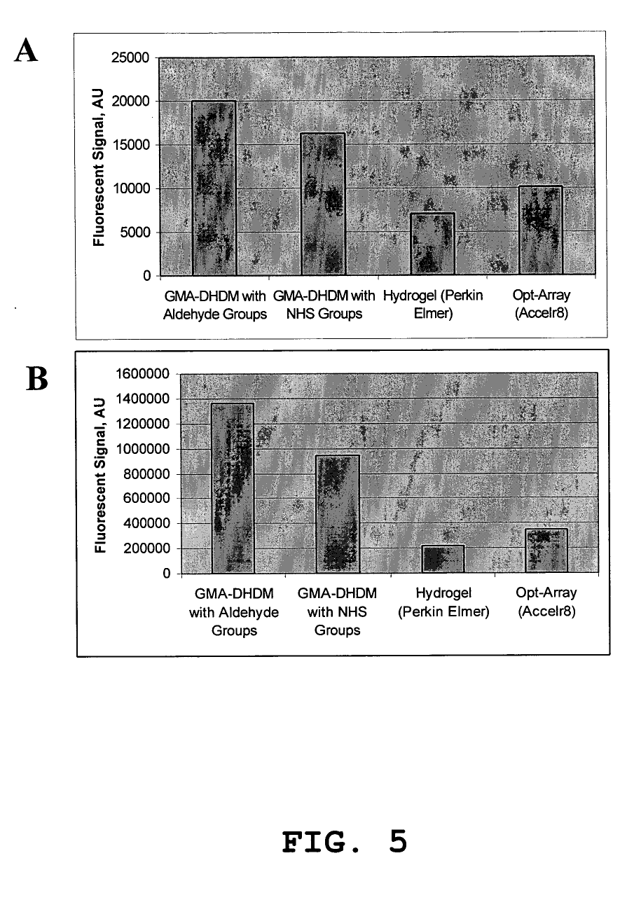

A biochip substrate produced with III, as described herein, was incubated for 3 h in 0.5 M boron trifluoride etherate in ethanol-water (9:1 v/v). The substrate was then washed with deionized ethanol for 5 minutes and then with water for 5 minutes. The substrate was then treated with 2% trifluoroacetic acid for 15 minutes and washed with deionized water for 5 minutes. Then the substrate was treated with 0.1 M sodium periodate for 30 minutes at room temperature to produce aldehyde groups (FIG. 5). After the treatment the substrate was dried.

The biochip substrates produced with I and II do not require any additional treatment after preparation of biochip substrates that are described in Examples 3-8. Biochip substrates produced with I or II contain N-hydroxy succinimide groups or isothiocyanate groups that are chemically-reactive towards amino groups. Thus, the biochip substrates produced with I and II do not require any additional treatment after preparation of biochip substrates that are described in Examples 3-8.

### Example 12

### Delivering Proteins or Oligonucleotides onto a Biochip Substrate

Protein solutions or solutions of oligonucleotides modified with amino groups were applied onto any of the substrates described in Examples 3-10 by a Quadrat II robot.

### Example 13

### Immobilization of Proteins or Oligonucleotides on the GMA Substrate with I or II

After the proteins or oligonucleotides were delivered as described in Example 11, the biochip substrates were incubated in a humid chamber for 12 hours to provide coupling of I or II incorporated into the polymer structure with the amino groups of either proteins or oligonucleotides modified with amino groups. The biochips were then treated either with 25 mM cystein chloride in 0.2 M carbonate buffer (pH 9.0) for 6 h or 0.1 M Tris-HCl (pH 9.0) for 24 h to activate the epoxide groups. The biochips were then washed with MilliQ water and air dried.

Epoxide groups that are present in GMA compositions are reactive towards amino groups.

### Example 14

### Immobilization of Proteins or Oligonucleotides on the GMA Substrate with III or GMA Substrates without I, II or III

After the proteins or oligonucleotides were delivered onto the GMA substrate with III (functional description in Examples 9 and 10)as described in Example 11, the biochip substrates were incubated in a humid chamber for 12 hours to provide coupling of aldehyde groups incorporated into the polymer structure with amino groups of either proteins or oligonucleotides modified with amino groups. The biochips were then incubated for 5 minutes in 0.1 M sodium borohydride solution to provide reduction of Schiff's base and convert remaining reactive aldehyde groups to hydroxide groups. The biochips were then washed with MilliQ water and air dried.

### Example 15

### Immobilization of Oligonucleotides or Proteins on the GMA Substrate with III or GMA substrates without I, II or III (Porane-Pyridine Method)

After the proteins or oligonucleotides were delivered onto the GMA substrate with III (functional description in Examples 9 and 10) as described in Example 11, the biochip substrates were incubated in a humid chamber containing vapors of borane-pyridine complex (Aldrich, St. Louis, Mo.) for 12 hours to provide coupling of aldehyde groups incorporated into the polymer structure with amino groups of oligonucleotides and reduction of the Schiff base formed in the vapors of pyridine-borane. The biochips were then incubated for 5 minutes in 0.1 M sodium borohydride solution to convert remaining reactive aldehyde groups to hydroxide groups. The biochips were then washed with MilliQ water and dried.

### Example 16

### Immobilization of Oligonucleotides on the GMA Substrate with III or GMA Substrates without I, II or III (Water-Saturated Chloroform Method)

After the proteins or oligonucleotides were delivered onto the GMA substrate with III (functional description in Examples 9 and 10) as described in Example 11, the biochip substrates were incubated under 0.1 M borane-pyridine in water-saturated chloroform for 12 hours to provide coupling of aldehyde groups incorporated into the polymer structure with amino groups of oligonucleotides and reduction of formed Schiff base. The biochips were then washed briefly with water and incubated for 5 minutes in 0.1 M sodium borohydride solution to convert the remaining reactive aldehyde groups to hydroxide groups. The biochips were then washed with MilliQ water and dried.

### Example 17

### Hybridization of Oligonucleotide Probes on Biochips with Substrates Described in Examples 4-9

2 mM solution of 20-base oligonucleotide containing 3′ amino group was used for loading (as described in Example 12) and immobilization (as described in Examples 13, 16) on the biochip substrates produced as described in Examples 3-8 and biochips produced using an acrylamide substrate

Hybridization with complementary Texas Red labeled oligonucleotides have been carried out for 12 hours the buffer containing 1 M guanidine isothiocyanate, 50 mM HEPES (pH 7.0), 5 mM EDTA. Fluorescent signal after hybridization have been recorded on a fluorescent microscope. FIG. 5 shows the hybridization signals recorded on different biochips.

### Example 18

### Comparative Binding of Texas Red Labeled Streptavidin on Biochips Produced with Different Substrates

Biotinylated BSA (100 mg/ml) (Pierce, Rockford, Ill.) in phosphate buffered saline buffer was used for loading (as described in Example 12) and immobilization (as described in example 14) on the GMA-DHDM biochip substrates with I and III produced as described in Example 5. The BSA was also loaded onto Hydrogel slide (Amersham, Piscataway, N.J.) and Opt-Array slides (Accelr8, Denver, Colo.) and immobilization was carried out according to recommendations from the manufacturers.

After the immobilization step, the biochips were washed in Washing Station (Telechem, Sunnyvale, Calif.) for 30 min with Tris buffered saline buffer containing 0.1% Tween-20; rinsed 3 times with Tris buffered saline buffer and then was incubated for 1 h in Tris buffered saline buffer (TBS) containing 1% BSA. Then the biochips were incubated in Super Block solution (Pierce, Rockford, Ill.) for 16 h at 4° C. After the incubation, the biochips were rinsed with MilliQ water and dried before the assay.

Biochips were treated with streptavidin-Texas Red conjugate (1 μg/ml and 10 ng/ml) in Tris buffered saline buffer (TBS) containing 1% BSA and 0.1% Tween-20. The incubation step of the assay on biochips were carried out in 20-μl incubation chambers (Grace Biolabs, Bend, Oreg.) at room temperature for 1.5 h. After the assay the biochips were rinsed 3 times with TBS containing 1% BSA and 0.1% Tween-20 and then washed for 15 min in TBS containing 1% BSA and 0.1% Tween-20 in Washing Staion, rinsed with MilliQ water and dried. Fluorescent signals from the biochips were recorded by Biolmager laser scanner (Packard Bioscience, Boston, Mass.).

FIG. 5 shows efficiency of binding for Streptavidin-Texas Conjugate with biotinylated BSA on GMA-DHDM and commercially available 3D biochip platforms.

### Example 19

### Comparative Study of Immunobinding on Different Biochip Substrates

Two mouse monoclonal antibodies, M421AE and M620E and Protein A (all at concentration 1.00 mg/ml) (Pierce) in phosphate buffered saline buffer were used for loading (as described in Example 12) on the GMA-EDMA and GMA-DHDM biochip substrates with III produced as described in Examples 4 and 5. The same proteins were also loaded onto a standard ANL polyacrylamide biochip substrate and SuperAldehyde substrate (Telechem). Each biochip contained 10 replicate loadings for each protein. Immobilization of the proteins was carried out as described in Example 14.

After the immobilization step, the biochips were washed in Washing Station (Telechem) for 30 min with Tris buffered saline buffer containing 0.1% Tween-20; rinsed 3 times with Tris buffered saline buffer and then was incubated for 1 h in Tris buffered saline buffer (TBS) containing 1% BSA. After the incubation, the biochips were rinsed with MilliQ water and dried before the assay.

Biochips were treated with goat antimouse-Texas Red conjugate (1 μg/ml) in Tris buffered saline buffer (TBS) containing 1% BSA and 0.1% Tween-20. The incubation step of the assay on biochips was carried out in 20-μl incubation chambers (Grace Biolabs) at room temperature for 1.5 h. After the assay the biochips were rinsed 3 times with TBS containing 1% BSA and 0.1% Tween-20 and then washed for 15 min in TBS containing 1% BSA and 0.1% Tween-20 in Washing Station, rinsed with MilliQ water and dried. Fluorescent signals from the biochips were recorded by ANL fluorescent microscope, and average values corresponding to each protein probe were calculated. Average fluorescent values for empty spots was calculated and then subtracted from the average values for spots with each protein probe for each biochip.

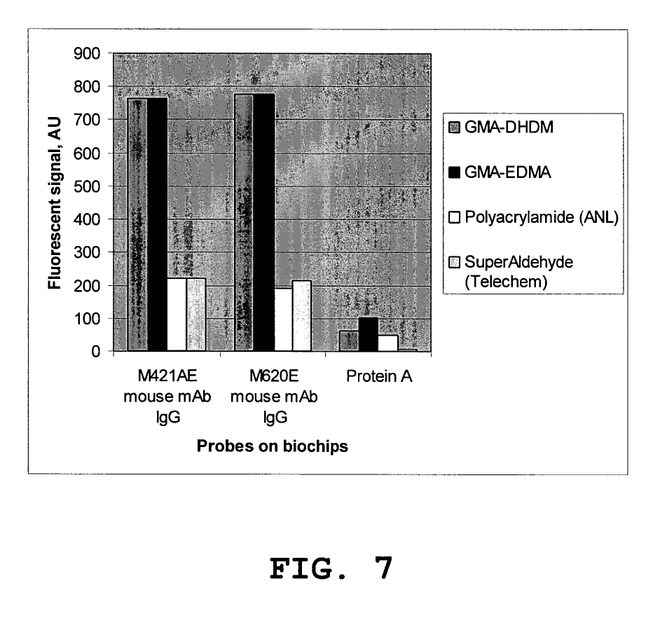

FIG. 7 shows efficiency of binding for goat anti-mouse-Texas Red conjugate with different protein probes on GMA-EDMA, GMA-DHDM, ANL polyacrylamide and SuperAldehyde substrates. Data suggests that ANL polyacrylamide substrate provide low efficiency for immunobinding that is approximately the same as on a planar substrate (SuperAldehyde) and noticeably less than on GMA-EDMA and GMA-DHDM substrates.

Preparation of Acrylamide Micro-Matrices by Photo-Polymerization

Preparation of glass slides
1. Immerse 10 glass slides in 10 M sodium hydroxide in a Wheaton glass-slide container (volume 150 ml) for 30 minutes.
2. Rinse with five changes of double-distilled water in a container.
3. Immerse 10 slides in concentrated sulfuric acid in container for 30 minutes
4. Rinse five times in double-distilled water and allow to stand in double distilled water for 5 minutes then rinse again.
5. Remove water drops with nitrogen stream. Dry for 1 h at 60° C.

Treatment of cleaned slide with Bind Silane.

Immerse slides in 3-(Trimethoxysilyl)propyl methacrylate and incubate for 40 h min at 37° C.

Rinse thoroughly with ethanol and then double-distilled water and dry under a nitrogen stream.

Preparation of solutions for aldehyde matrices
1. Composition of 5% polyacrylamide solution
   0.5 ml 40% Acrylamide/Bis solution (19:1)
   1.82 ml 0.2M sodium phosphate buffer (consists of equal volumes of 0.2M sodium phosphate monobasic monohydrate and 0.2M sodium phosphate dibasic anhydrous pH=6.8, store at 4° C.).
   1.6 ml glycerol
   0.08 ml monomer I solution (N-(5,6-di-O-isopropylidene)hexyl acrylamide). For monomer I solution add 1 ml MilliQ water to aliquot of monomer I stock (25 mg) located in −80° C. freezer. Aliquot and store these at −20° C. for approximately 1 month.
2. filter.
3. Prepare solution weekly and store at 4° C. Allow solution to reach room temperature before use.

Assembly of gel-casting cassette.
1. Clean mask surface with ethanol.
2. Rinse thoroughly with distilled water stream rubbing briskly with lint-free tissue.
3. Dry under a nitrogen stream.
4. Place spacers (audio tape film) on chrome side of mask; two spacers from both sides and one in the center.
5. Place slide over mask and spacers with treated surface facing mask.
6. Clamp in place.

Photo-polymerization (optimized for 4-cluster mask).

Prepare mixture: 100 μl of above polyacrylamide solution
0.4 μl Methylene blue (0.04%)
1.2 μl TEMED
Vortex 3 seconds
Degas solution 3 min

Pipette mixture between the slide and the mask allowing the solution to move between the space by capillary action. Take care that air does not enter the pipette or space. Pipette off excess solution.

Turn cassette over so that glass slide is underneath the mask. Place in Oriel chamber.

Irradiate for 300 sec.

Carefully disassemble the cassette under water using the point of a scalpel to separate the slide and mask ( the slide floats free without pressure being placed on the gel elements.) Take care not to scratch mask.

Rinse 30 seconds under running distilled water and soak in distilled water for 15 minutes

Air dry in a laminar-flow hood

Keep in dust-free slide box at room temperature. Matrices can be kept for at least 1 year.

Procedure for activation (deprotection) of aldehyde matrices
1. Place matrix in 2% trifluoro-acetic acid for 10 minutes at room temperature (prepare fresh solution after every 10 microchips).
2. Rinse well (5 or 6 times) with filtered distilled water for 1 min
3. Wash in distilled water×3 times then leave 3-5 mins in last rinse and dry 20 min in air.
4. Put slide into Repel Silane™ (use fresh solution for each treatment) for one minute.
5. Wash with acetone or dichloromethane (15 sec) and then thoroughly with tap—distilled water (15 sec under stream).
6. Load oligonucleotides.
7. Put microchip into freshly prepared solution of pyridine-borane complex in chloroform (0.1M)(80 ml chloroform/1 ml pyridine borane) and cover chloroform layer with water; (approx ¼ inch)hold 12 hours at room temperature (O.N.)
8. Wash microchip with distilled water.
9. Place microchip into 0.1M sodium borohydride on microchip for 20 min.
10. Wash with distilled water 1 min.
11. Heat microchip in 0.1×SSPE with 0.1% SDS at 60° C. for 1 h (50 ml).
12. Wash biochip in Hybridization Station for 15 min on a stirrer.
13. Wash with distilled water 1 min.
14. Dry microchip in a dust-free environment in the air for 20 min.
15. The chip is now ready for hybridization. The chip could be kept at room temperature.

Standardized Sources of Chemicals and Equipment
1. DEPC-Treated Water (Ambion, cat #9920)
2. 0.5M EDTA, pH 8.0 (Ambion, cat #9260G)
3. Eppendorf Centrifuge 5417C (Fisher, cat #05-406-11)
4. Eppendorf microcentrifuge tubes, 1.5 ml (Fisher, cat #05-402-24B)
5. Acetone (Sigma, cat #A4206)
6. Guanidine Thiocyanate (Fisher, cat #BP221-1)
7. 1M HEPES (Sigma, cat #H4034)
8. Hybridization chamber: Probe-Clip Press-Seal Incubation Chamber (Sigma, cat #Z36,157-7)
9. Kimwipes (Fisher, cat #06-666A)
10. 20×SSPE (Sigma, cat #S2015)
11. Tween 20 (Fisher, cat #BP337-100)
12. Imaging Chamber (Sigma, cat #Z36,585-8)
13. Ultrafree-MC 0.45 μm filter unit (Millipore, cat #UFC30HVNB)
14. Triton X-100 (Sigma, cat #T9284)
15. Ethyl Alcohol, absolute 200 proof, 99.5%, A.C.S. reagent (Aldrich, cat #45,984-4)
16. QIAquick PCR Purification Kit (50) (Qiagen, cat #28104)
17. Taq DNA Polymerase (includes 10× PCR reaction buffer) (Amersham Pharmacia Biotech, cat #T0303Z)
18. PCR Nucleotide Mix: PCR nucleotide mix (10 mM each dATP, dCTP, dGTP, DTTP) (Amersham Pharmacia Biotech, cat #US77212)
19. Sonicated Salmon Sperm DNA, Phenol Extracted (Amersham Pharmacia Biotech, cat #27-4565-01)
20. Albumin from bovine serum, 20 mg/ml in water (Sigma, cat #B8667)
21. Luer Lok syringe, 60 cc/2 oz, B-D (Fisher cat #14-823-2D)

Millex-GN 0.20 filter units (Millipore, cat #SLGN025NS)

Documents Cited

The documents cited herein are incorporated by reference to the extent they relate protocols, materials or methods related to the present invention.

Horak, D et al., (1993) The Effect of Polymeric Porogen on the Properties of Macroporous Poly(Glycidyl Methacrylate-co-Ethylene Dimethacrylate). Polymer 34, 3481-3489.

Jovanovic et al., (1994) The influence of Inert Component Composition on the Porous Structure of Glycidyl Methacrylate/Ethylene Glycol Dimethacrylate Copolymers. Die Angewndte makromolekulare Chemie 219, 161-168.

H. J. Naghash, 0. Okay, and Y. Yagci. 1997. Gel formation by chain-crosslinking photopolymerization of methyl methacrylate and ethylene glycol dimethacrylate. Polymer 38(5): 1187-1196.

Svec et al., (1995) Kinetic Control of Pore Formation in macroporous Polymers. Formation of “Molded” Porous Materials with High Flow Characteristics for Separations or Catalysis. Chem. Mater. 7, 707-715.

1 A method of making a microarray with a macroporous polymer substrate, the method comprising:
(a) obtaining a macroporous polymer substrate; and
(b) coating a surface with the substrate.

2 The method of claim 1, wherein the macroporous polymer substrate is synthesized by a method comprising:
(a) obtaining methacrylates;
(b) mixing the methacrylates in the presence of a porogenic solvent; and
(c) initiating polymerization to form a macroporous polymer substrate.

3 The method of claim 2, further comprising:
(a) obtaining at least one immobilization chemical for immobilization of biomolecules to the microarray; and
(b) adding the immobilization chemical to the macroporous polymer substrate.

4 The method of claim 3, wherein the macroporous polymer substrate is applied to a surface selected from the group consisting of glass, metal, silane, silicone, and plastics with vinyl.

5 The method of claim 1, wherein the biomolecules are selected from the groups consisting of DNA, RNA, peptides, proteins, lipids, lipopolysaccharides, antibodies, and peptide mimetics.

6 The method of claim 2, wherein the methacrylates are selected from the group consisting of monofunctional methacrylates and polyfunctional methacrylates.

7 The method of claim 6, wherein the monofunctional methacrylates are selected from the group consisting of alkyl-, epoxyalkyl-, hydroxyalkyl-, and polyoxyalkyl ethers of methacrylic acid.

8 The method of claim 6, wherein the polyfunctional methacrylates are selected from the group consisting of dimethacrylates of ethylene glycol, di-, tri, and tetramethacrylates of polyols.

9 The method of claim 2, wherein the methacrylates are selected from the group consisting of GMA, HEMA, EDMA, and DHDM.

10 The method of claim 2, wherein the porogenic solvent is an aromatic alcohol.

11 The method of claim 10, wherein the aromatic alcohol is selected from the group consisting of cyclohexanol and dodecanol.

12 The method of claim 2, wherein the porogenic solvent is an aliphatic alcohol.

13 The method of claim 2, wherein the porogenic solvent is an aromatic alkyl derivative.

14 The method of claim 3, wherein the immobilization chemical is derivatized to include functional groups selected from the group consisting of aldehydes, succinimides and isothiocyanates.

15 The method of claim 3, wherein the immobilization chemical is selected from the group consisting of N-(methacryloyl)aminocaproic acid N-hydroxysuccinimide ether, 4-isothiocyanate-N-(methacryloyl)benzylamine, and N-(5,6-di-O-isopropylidene)hexyl acrylamide.

16 A method of analyzing molecular interactions, the method comprising the steps of:
(a) immobilizing at least one probe molecule to a macroporous polymer substrate comprising a monofunctional methacrylate, a polyfunctional methacrylate, and a solvent;
(b) obtaining at least one analyte molecule;
(c) providing suitable conditions for the probe-analyte interaction; and
(d) measuring signal from the interaction.

17 The method of claim 16, wherein the probe molecule is selected from the group consisting of antibodies, peptides, proteins, and DNA.

18 The method of claim 16, wherein the analyte is derived from a biological sample.

19 The method of claim 16, wherein the probe-analyte interaction is an antigen-antibody interaction.

20 The method of claim 16, wherein the probe-analyte interaction is a nucleic acid hybridization.

21 A microarray with a macroporous polymer substrate comprising:
a monofunctional methacrylate,
a polyfunctional methacrylate or a mixture thereof;
an immobilization chemical; and
a porogenic solvent.

22 The macroporous polymer substrate of claim 21, wherein the monofunctional methacrylates are selected from the group consisting of glycidyl methacrylate, and 2-hydroxyethyl methacrylate.

23 The macroporous polymer substrate of claim 21, wherein the polyfunctional methacrylate is selected from the group consisting of ethylene dimethacrylate, and 2,3-dihydroxybutane-1,4-diyl dimethacrylate.

24 The macroporous polymer substrate of claim 21, wherein the immobilization chemical is selected from the group consisting of N-(methacryloyl)aminocaproic acid N-hydroxy succinimide ether, 4-isothiocyanate-N-(methacryloyl)benzylamine, and N-(5,6-di-O-isopropylidene)hexyl acrylamide.

25 The macroporous polymer substrate of claim 21, wherein the porogenic solvent is selected from the group consisting of cyclohexanol and dodecanol.

26 The macroporous polymer substrate of claim 21 comprising 4-30% GMA, 2-20% of EDMA, 0-5% MAAHSE, 48-60% cyclohexanol, and 0-12% dodecanol.

27 The macroporous polymer substrate of claim 21 comprising 4-30% GMA, 2-20% of DHDM, 0-5% ITCMBA, 48-60% cyclohexanol, and 0-12% dodecanol.

28 The macroporous polymer substrate of claim 21 comprising 4-30% GMA, 2-20% of EDMA, 0-5% ITCMBA, 48-60% cyclohexanol, and 0-12% dodecanol.

29 The macroporous polymer substrate of claim 21 comprising 4-30% GMA, 2-20% of DHDM, 0-5% MAAHSE, 48-60% cyclohexanol, and 0-12% dodecanol.

30 The macroporous polymer substrate of claim 21 comprising 4-30% HEMA, 2-20% of EDMA, 0-5% MAAHSE, 48-60% cyclohexanol, and 0-12% dodecanol.

31 The macroporous polymer substrate of claim 21 comprising 4-30% HEMA, 2-20% of DHDM, 0-5% ITCMBA, 48-60% cyclohexanol, and 0-12% dodecanol.

32 The macroporous polymer substrate of claim 21 comprising 4-30% HEMA, 2-20% of EDMA, 0-5% ITCMBA, 48-60% cyclohexanol, and 0-12% dodecanol.

33 The macroporous polymer substrate of claim 21 comprising 4-30% HEMA, 16% of DHDM, 0-5% MAAHSE, 48-60% cyclohexanol, and 0-12% dodecanol.

34 A macroporous polymer substrate comprising:
a monofunctional methacrylate;
a polyfunctional methacrylate; and
a solvent.

35 The macroporous polymer substrate of claim 34, wherein the monofunctional methacrylate is selected from the group consisting of glycidyl methacrylate, and 2-hydroxyethyl methacrylate.

36 The macroporous polymer substrate of claim 34, wherein the polyfunctional methacrylate is selected from the group consisting of ethylene dimethacrylate, and 2,3-dihydroxybutane-1,4-diyl dimethacrylate.

37 The macroporous polymer substrate of claim 34, wherein the solvent is selected from the group consisting of cyclohexanol and dodecanol.
